(19) United States
(12) Patent Application Publication
(10) Pub. No.: US 2008/0241200 A1
(43) Pub. Date: Oct. 2, 2008

(54) Cosmetic skin care system

(76) Inventor: Marcy Elizabeth Sojka, Neenah, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/800,692

(22) Filed: May 7, 2007

Related U.S. Application Data
(60) Provisional application No. 60/921,005, filed on Mar. 30, 2007.

(51) Int. Cl. A61K 8/30; A61B 17/50; A61Q 19/00; A61K 36/02

(52) U.S. Cl. 424/401; 424/195.17; 606/131

(57) ABSTRACT

A skin care cosmetic system that includes components in unit doses. Each system includes a substrate that is formed into a patch or mask, or a garment having a volume, for example, a sock, glove, or a sleeve. The substrate is treated with a moisture-containing formulation. Other system components may be unit doses of finishing lotion or skin preparation formulations or devices.

### CROSS-REFERENCE TO RELATED APPLICATION

The invention of the present application claims the benefit of provisional U.S. patent application Ser. No. 60/921,005 filed on Mar. 30, 2007.

### BACKGROUND OF THE INVENTION

In a health-conscious, youth-oriented society, healthy looking skin is desired by many. One way to get healthy looking skin is to obtain services at a spa. However, time and cost prevent most persons from visiting a spa as often as might be needed to maintain or achieve a desired result. Thus, most people obtain the majority of skin care components from a retail store.

A visit to the cosmetic aisle of major retailers will reveal a large selection of lotions, creams, cleansers, and spa articles. There are many items to choose from if one desires to purchase components for a home spa-treatment experience. Skin components are available that cleanse, exfoliate, tone, moisturize, lighten, etc. In fact there are so many components, it is difficult to choose what to purchase, and perhaps, how to use certain components together to achieve a desired result.

One solution is to purchase a line of home spa-treatment components from a single manufacturer or brand. Many component lines are sold which contain components that are intended to complement one another. For instance, some companies sell soap, toner, and moisturizers that are intended to work together in a step-by-step system to achieve clean, hydrated skin. Unfortunately, one has to purchase several components to achieve the advertised results. Each component may be used for several applications, and therefore, may mean a significant investment in a line of component that the purchaser may not enjoy after trying.

Another problem with the purchase of several components for step-by-step use is that the collection can be inconvenient to use when traveling. People often enjoy taking their favorite skin care preparations on vacation. However, with the weight and security restrictions imposed on persons that travel by air, it may not be feasible to take such components along on vacation. At the very least, they can add an undesirable amount of weight to a bag, and take up precious space.

Finally, when one finds a step-by-step, cosmetic skin care system that works well, he or she may want to share all or part of the cosmetic system with a friend. However, this may be inconvenient, or not feasible, short of loaning a person all of one's personal inventory of the cosmetic system. Loaning cosmetic components to others can be very unsanitary. Further, the loaner may not have the cosmetic system on hand when he or she wants to use it.

In light of the foregoing problems and issues discussed above, there remains a need for a step-by-step cosmetic skin care system that is easy and sanitary to share. It is also desired that the system be relatively inexpensive. It is further desired that the system is easy to transport and store. Finally, it is desired that the system is relatively easy to use to facilitate convenient pampering.

### SUMMARY OF THE INVENTION

In one aspect of the present invention, there is provided a cosmetic skin care system. The device includes a cosmetic system for the skin which includes a unit dose of a skin preparation compound in a first container, a unit dose of a substrate comprising a sheet material with a skin care composition disposed thereon in a second container, and a unit dose of a finishing lotion in a third container. The first, second, and third container are combined into a common package.

In another aspect of the present invention, there is provided a method of packaging a cosmetic system for the skin of a targeted body part. The method steps include: a. disposing a unit dose of an exfoliation device in a first container to create an exfoliation component; b. disposing a unit dose of a finishing lotion in a second container to create a lotion component; and c. disposing a unit dose of a substrate having a volume adapted to receive a body part into a third container to create a substrate component. The lotion component, the exfoliation component, and the substrate component together form a first component set which is disposed into a common container.

In still another aspect of the present invention, there is provided a cosmetic system for the skin including a skin exfoliation component, a substrate comprising a sheet material and a hydrogel disposed thereon, and a finishing lotion. The skin exfoliation component, the substrate, the lotion, and instructions for use of the same are combined into a common package.

Still other features of the invention will be in part apparent and in part pointed out hereinafter as well as better understood by practice of the invention. It is to be understood that both the foregoing general description and the following detailed description are exemplary and are intended to provide further explanation of the invention claimed. The accompanying drawings, which are incorporated in and constitute part of this specification, are included to illustrate and provide a further understanding of the cosmetic skin care system.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a front perspective view of one embodiment of a system of the present invention.

FIG. 2 is a front elevational view of one embodiment of a substrate of the present invention in the form of a face mask, the mask having a unitary configuration.

FIG. 3 is a front elevational view another embodiment of a substrate of the present invention in the form of a face mask, the mask configured to cover a relatively small portion of the face.

FIG. 4 is a front elevational view yet another embodiment of a substrate of the present invention in the form of a face mask, the mask having a multi-part configuration.

FIG. 5 is a bottom elevational view of an embodiment of the substrate configured to cover other portions of the body, in particular the sole of the foot.

FIG. 6 is a rear elevational view of another embodiment of a substrate configured to cup the heel of the foot.

FIG. 7 is a side elevational view of the substrate shown in FIG. 6

FIG. 8 is a front perspective view of one embodiment of an exfolient device of the present invention.

FIG. 9 is a side perspective view of another embodiment of the substrate configured into a sock.

FIG. 10 is a front elevational view of another embodiment of the substrate configured into a glove.

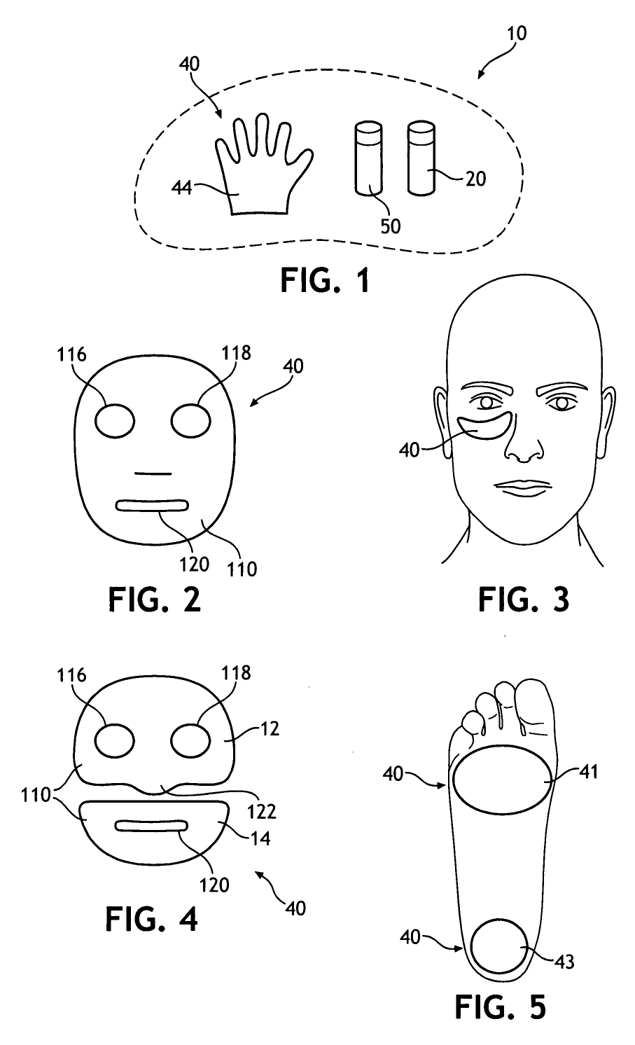

### DETAILED DESCRIPTION OF THE PRESENT INVENTION

Reference will now be made in detail to embodiments of the invention, one or more examples of which are illustrated in the drawings. Each example is provided by way of explanation of the invention, and not meant as a limitation of the invention. For example, features illustrated or described as part of one embodiment can be used with another embodiment to yield a third embodiment. It is intended that the present invention include these and other modifications and variations.

The term “water” as used herein is intended to mean tap water, spring water, mineral water, or distilled water.

The term “hydrogel” as used herein is intended to mean a network of polymer chains that are water soluble. Hydrogels are superabsorbent (they can contain over 99% water) natural or synthetic polymers.

The term “skin preparation compound” as used herein is a substance used to prepare the skin prior to applying a substrate of the present invention to the skin. Examples of skin preparation compounds include cleansers such as soap and the like, exfoliates, and wetting agents. Such examples are not intended to limit the scope of the present invention.

The term “finishing lotion” as used herein is a substance that is applied to the skin after the substrate has been applied to the skin and removed. Examples of finishing lotion may include under-eye treatment, lotions for preventing loss of moisture, and the like. Such examples are not intended to limit the scope of the present invention.

The term “unit dose” as used herein is intended to mean an effective amount of substance, or an effective number of articles, adequate for a single session. By way of example, a unit dose of moisturizing lotion for the face may be defined as enough lotion for a person to coat the face. The surface area of the face may vary somewhat from person to person. Thus, a person using a unit dose may have excess lotion left over. It is desirable to provide enough lotion that even the above-average sized face will have an effective amount of coverage. In another example, a unit dose of socks may be two socks, one for each foot.

Very generally, the present invention is a step-by-step cosmetic skin-care system that includes several articles which may be used in a particular order; one of the articles includes a substrate that is configured to fit a particular part of the body. The other articles may be a cosmetic formulation and/or a cosmetic device including, but not limited to skin preparation components and finishing lotions. An effective amount of each article that is suitable for a single use, in whatever form (liquid, solid, and/or device), may be separately packaged to create separate “system components.” Each system component may be combined into a common package for display and sale in a retail setting, either virtual or actual. Instructions indicating the order of use for each system component is used may be included as a separate brochure, or printed directly on the common package or each of the system components.

The common package used to contain the system components for sale may be any package type as is known in the art, e.g. a bag, box, can, basket, glass or plastic jar, blister pack, and the like. The package may be intended for disposal after the system component are used, or may be reusable to store other items once the system components are used. One representative example of a package that may be hold the cosmetic system of the present invention is found in commonly owned U.S. patent application entitled “PACKAGE WITH EXPOSED PULL,” filed Mar. 5, 2007, docket number 64356874.

There are several advantages presented by the cosmetic system of the present invention. First, each component is either used up or discarded after the package is opened, with little or no waste. This is far more sanitary than using full- or bulk-sized components. After some time, spoilage and contamination of full- or bulk-sized components is a concern, and substantial amounts of the component product may need to be tossed out, an act that may not be economically beneficial. Further, the unit doses of each component make it very easy to transport on vacation, or to share all or part of the system with a friend. Such packets also take up little space at home, or other storage places. In addition, there is no large investment into a set of components that may not work for particular consumers. Moreover, the cosmetic system is easy to purchase and use as there is no need for measuring. Everything needed for a one-time or single beauty-session use is located in one package, eliminating the time it takes at the store to find everything one might need. The consumer is assured that all the components are compatible and work together without an adverse reaction between the components. The system facilitates the pampering of skin away from the spa setting, so that consumers can relax and enjoy skin care any time.

There are many possible combinations of multi-step cosmetic skin care systems, and several exemplary embodiments are described herein in the next section. The specific formulations and devices or appliances listed in the following embodiments of the present invention are described more fully under the section entitled “Formulations and Devices.”

Tables 1-3 below show not only the following representative embodiment of the present invention, but other possible combinations thereof. Regardless of the many possible combinations, it is desired that there are at least three components in any given system so that the user has the feelings of an actual spa experience. There are many cosmetic treatments that only utilize two steps, but that is not as relaxing as having three or more steps. The more ritualistic this or any of the cosmetic treatments are, the more “spa-like” and relaxing the experience may be.

### Embodiment One

One exemplary embodiment of the present invention 10 is a skin care system for the face. Desirably, there are at least three components included in the system. One component is a substrate that is applied to the skin in the form of a mask, (“substrate component 40”). The mask substrate 40 may be a full facial mask as seen in FIG. 2, or a partial mask, such as an under eye patch or a mask that fits a particular region of the face or even the neck. See FIGS. 3 and 4. Desirably, the full facial mask is split into two portions, an upper portion 12 and a lower portion 14. The advantage of two portions is that it may be easier to apply, and allows the possibility of different cosmetic formulations to be applied to the different sections. In addition, it more aesthetic as it looks less like a Halloween mask. Of course, the mask may be split into side-by-side portions, or further subdivided into more than two portions. Regardless of the mask size, the purpose of the substrate component is to impart moisture, emollients, humectants or other skin conditioning agents to the skin without mechanically removing hair from the skin. Mechanical removal of hair is not desired in this application because it is typically painful and could as a result, hinder relaxation intended from use of system 10.

Other possible components for the skin care system for the face include a skin preparation article such as a wetting agent (“wetting component 20”) and/or an exfolient as described herein (exfoliation component 30). The exfolient may be a device, a compound, or a combination of both. Yet other possible components include one or more finishing lotions such as lotion component 50, a skin lightening agent (“skin lightening component 60”), and/or possibly a toner (“toner component 70”). Each of these components is described in more detail herein.

TABLE 1
Possible combinations of cosmetic systems for the face.

| Wetting | Exfollient | Substrate | Lotion | Skin |
|---|---|---|---|---|
| component | component | component | component | lightening |
| 20 | 30 | 40 | 50 | component 60 |
| X | X | X | | |
| X | | X | X | |
| X | | X | | X |
| | X | X | X | |
| | X | X | | X |
| | | X | X | X |
| X | X | X | X | |
| | X | X | X | X |
| X | | X | X | X |
| X | X | X | | X |
| X | X | X | X | X |

Instructions included with this particular embodiment of the present invention 10 may indicate that the wetting component 20 is used as an optional first step. Next, the exfoliation component 30 may be used as a second step. In the alternative, the exfoliation and wetting steps may be combined as the process of exfoliation commonly requires water or other liquids to be used in conjunction with the exfoliation device or compound. The substrate component 40 may be used as a third step. The skin lightening component 60 may be used as a fourth step. Finally, the lotion component 50 may be used as a final step. It is contemplated that other steps might be included in any combination of the system for the face, such as an optional toning step using a toner component 70. It is further contemplated that the steps may occur in a different order, and this particular order is not meant to limit the scope of the invention.

### Embodiment Two

Another exemplary embodiment of the present invention 10 is a skin care system for the feet. Desirably there are at least three components included in the system. One component is a substrate formed into a bootie or sock (“substrate component 40”). In the alternative, rather than providing a bootie or sock, a patch component may be provided. The patch may be shaped to fit on the heel of the foot, or other problematic areas of the foot such as the area 41 between the toes and the metatarsal arch, or the sole region 43 of the heel, see FIG. 4.

Yet another component in the skin care system for the feet is a finishing lotion (“lotion component 50”). Another component may be a “thermal component 80” that can heat or cool the skin, and that may be used in addition to, or in replace of the lotion component 50. Most desirably, an additional skin preparation component is included in the system, such as a wetting agent for the skin (“wetting component 20”). Another optional component that may be included with any of the exemplary systems shown in Table 2 is a nail conditioner component 90. Please refer to Table 2 for other possible combinations of the skin care system for feet.

TABLE 2
Possible combinations of cosmetic systems for the feet.

| | Exfollient | Substrate | Lotion | Thermal |
|---|---|---|---|---|
| Wetting | component | component | component | component |
| component 20 | 30 | 40 | 50 | 80 |
| X | X | X | | |
| X | | X | X | |
| X | | X | | X |
| | X | X | X | |
| | X | X | | X |
| | | X | X | X |
| X | X | X | X | |
| | X | X | X | X |
| X | | X | X | X |
| X | X | X | | X |
| X | X | X | X | X |

Any instructions included with this particular embodiment of the present invention 10 may indicate that wetting component 20 is used as a first step, if present. Next, the exfoliation component may be used as a second step. The substrate component 30 may be used as a third step. A thermal component 80 may be used as a fourth step. Finally, the lotion component 50 and optional nail conditioner 90 may be used as a combined final step. It is contemplated that other steps might be included in the system, such as a nail treatment step, e.g. a nail conditioner. It is further contemplated that the steps may occur in a different order, and this particular order is not meant to limit the scope of the invention.

### Embodiment Three

Yet another exemplary embodiment of the present invention 10 is a skin care system for the hands. Desirably there are at least three components included in the system. One skin preparation component exfoliates the skin (exfoliation component 30). Another component may include a substrate that is applied to the skin in the form of a glove or patch (“substrate component 40”). Yet another component is a finishing lotion for application to the skin (“lotion component 50”). Most desirably, a fourth component is included in the system, such as a wetting component 20, or a skin lightening component 60. Optional components may further include a cuticle and/or nail conditioner 90. Refer to Table 3 for other possible combinations of a skin care system 10 for the hands.

TABLE 2
Possible combinations of cosmetic systems for the hands.

| Wetting | Exfollient | Substrate | Lotion | Skin |
|---|---|---|---|---|
| component | component | component | component | lightening |
| 20 | 30 | 40 | 50 | component 60 |
| X | X | X | | |
| X | | X | X | |
| X | | X | | X |
| | X | X | X | |
| | X | X | | X |
| | | X | X | X |
| X | X | X | X | |
| | X | X | X | X |
| X | | X | X | X |
| X | X | X | | X |
| X | X | X | X | X |

Any instructions included with this particular embodiment of the present invention 10 may indicate that wetting component 20 is used as a first step, if present. Next, the exfoliation component 30 may be used as a second step. The substrate component 40 may be used as a third step. Finally, the lotion component 50 may be used as a final step. It is contemplated that other steps might be included in the system, such as a cuticle or nail preparation step. It is further contemplated that the steps may occur in a different order, and this particular order is not meant to limit the scope of the invention.

### Formulations and Devices

Each of the components of the above noted exemplary systems are described in more detail below.

### Wetting Component 20:

The purpose of wetting component 20 may be to prepare the skin for exfoliation. The wetting component 20 can clean skin prior to the exfoliation step by removing cosmetics or excess natural oils on the skin so that there is less of a barrier between the exfoliation device 30 and the skin. At the very least, the wetting agent hydrates the outer layers of the skin, making the skins cells larger and thus easier to remove mechanically with an exfoliation device. Many types of formulations as are known in the art are capable of hydrating and/or cleansing the skin. The wetting component 20 may be omitted, and the skin wetted with water prior to the exfoliation step.

One exemplary wetting component is a soap or detergent. Used with water, soap and detergent are capable of removing from the skin, excess skin oil and cosmetics such as foundation, blush, and powder. Examples of soap include: glycerin soap, hard-milled soap, castile soap, and the like. Examples of detergent include cleansers that contain ingredients such as sodium trideceth sulfate, cocamidopropyl hydroxysultaine, ammonium laureth sulfate, PEG-80 sorbitan laurate, disodium lauroamphodiacetate, glycol distearate, disodium oleamido MEA-sulfosuccinate, sodium caproyl lactylate, sodium lauroyl lactylate, polysorbate 20, sodium laureth-13 carboxylate.

Another exemplary wetting component is an effervescent tablet. The effervescent tablet is used with water to create a bubbly, aqueous soaking solution. One type of effervescent tablet includes sodium bicarbonate and citric acid. When added to water which acts as a catalyst, these ingredients react to create a gas. Many effervescent tablet formulations exist in the prior art, including that found in U.S. Pat. No. 5,997,901 issued to Mills. Other art includes commonly owned patent application U.S. Ser. No. 11/638,938, filed Dec. 13, 2006, by Druecke et al.; incorporated by reference to the extent it is compatible with the present invention.

In one representative example of the present invention, the effervescent tablet is manufactured from the following components: sodium bicarbonate; citric acid; sodium sulfate; sodium carbonate; water, one or more dyes such as titanium dioxide or blue 1 and yellow 5 (all optional); cellulose gum; and a fragrance (also optional).

Yet another exemplary wetting component 20 is a bath salt. Bath salts contain one or more of the following: Epsom salt, sea salt, baking soda, and the like. In addition, glycerin and/or a fragrance may be added to the bath salt. Bath salts are added to water used for soaking or bathing.

### Exfoliation Component 30:

Exfoliation is a cosmetic technique aimed at improving the skin's appearance by removing dead skin cells from the surface of the skin. Thus, the purpose of exfoliation component 30 is to slough dead outer skin cells from the skin, mainly from the stratum corneum layer. Removing excess dead skin cells may reveal the younger and healthier-looking skin underneath.

Exfoliation may be achieved through either mechanical or chemical methods. Mechanical exfoliants include polymer micro-beads, rice particles, crushed nut shells or fruit pits (e.g. peach pit shells, almond shells), sugar, salt, man-made or other types crystals, pumice, silica and abrasive substrates. Chemical exfoliants include chemical peels containing salicylic acid, glycolic acid, fruit enzymes, citric acid, or malic acid.

In one representative embodiment of the present invention, a mechanical exfoliation preparation, includes the following types of ingredients: abrasive material, surfactants, emulsifiers, skin conditioning agents, cleansing agent, an emollient, preservatives, a ph adjuster, moisturizer, and botanical extracts.

TABLE 4
Representative example of exfoliation compound.

| Component | Weight | | |
|---|---|---|---|
| (INCI Name) | % | Supplier | Trade Name |
| Algae Extract | | Active | Actiphyte of Algae |
| | | Organics | BG50P |
| Boswellia Serrata | | Active | Actiphyte of |
| Resin Extract | | Organics | Boswellia BG50P |
| Carthamus Tinctorium | | Cognis | High Oleic Safflower |
| (hybrid Safflower) | | | Oil |
| Seed Oil | | | |
| Cetearyl Alcohol | | Protameen | Cetyl Stearyl Alcohol |
| | | | 30/70 |
| Citric Acid | | Jungbunzlauer | Citric Acid |
| Fragrance | | Firmenich | Firmenich 178470B |
| Glycerin | | Hesego | Glycerin 99.7% |
| | | | Kosher |
| Glyceryl Stearate | | Stepan | Kessco GMS-SE |
| Glyceryl Stearate | | UPI | Unitolate 165C |
| PEG-100 Stearate | | | |
| Hydroxypropyl Guar | | Aqualon | N-Hance HP-40 |
| Methyl Gluceth-20 | | Alzo | Aloxe MG-20 |
| Oryza Sativa (Rice) | 5.00 | Sage 5 Foods | Rice Ground |
| Bran | | | RF-L00030 |
| Phenoxyethanol | | McIntyre | Paragon MEPB |
| Methylparaben | | | |
| Ethylparaben | | | |
| Propylparaben | | | |
| Butylparaben | | | |
| Potassium Hydroxide | | Oxychem | Potassium |
| | | | Hydroxide/KOH |
| Rice Hulls | 1.00 | Midwest Grain | Aqua Pro II RH |
| Sodium Cocyl | | Fintex | Tauronal I-78C |
| Isethionate | | | |
| Sodium Methyl | | Finetex | Tauranol WS |
| Cocoyl Taurate | | | Concentrate |
| Stearic Acid | | Pan Century | Vegetable Stearic |
| | | Oleochemicals | Acid, Triple Pressed |
| Water | 64.88 | n/a | n/a |

This preparation may be on most parts of the body, and may be desirable for use on the face and hands.

In another embodiment of the present invention, the exfolient may be a basic blend of an abrasive material, a preservative, and a solvent such as water. In yet another embodiment of the present invention, another mechanical exfoliation device includes a buff appliance or device, or buff 32, as seen in FIG. 8. The buff 32 may be constructed from synthetic and/or natural fibers. One representative example of a buff is an interbonded fibrous layer 34 having three-dimensional contours 36 on both faces 38 of the layer. The contours 36 facilitate exfoliation and/or stimulation and/or gentle abrasion of skin. Furthermore, the contours facilitate contact and attachment to a second interbonded fibrous layer. The second interbonded fibrous layer may include a high-loft material capable of gently cleaning skin, holding liquid, and generating lather. See commonly owned patent application, entitled “FOOT EXFOLIATING BUFF,” U.S. Ser. No. 11/265,391, filed Nov. 1, 2005, incorporated herein to the extent it does not conflict with the present invention.

### Substrate Component 40:

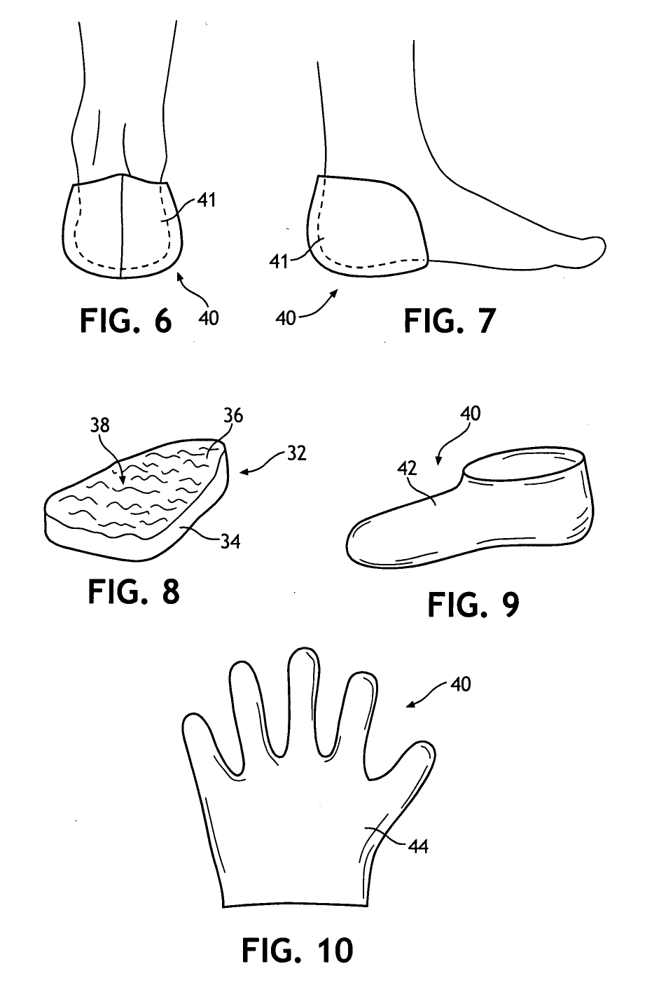

Referring to FIGS. 2-7, the primary purpose of substrate component 40 may be to place a hydrating compound against the skin, such as a hydrogel or moisturizing compound that that contains ingredients which cause the skin to appear smoother, possibly with less fine lines. In one embodiment of the present invention, a substrate component 40 is a face mask that is either a unitary mask, or a multi-part mask. In another embodiment of the present invention, the substrate component 40 is a bootie or sock 42 for wearing on the foot. In yet another embodiment of the present invention, the substrate component 40 is a glove 44 for wearing on the hand. A further embodiment shows a heel cup 41. Other substrate configurations are contemplated, such as an under eye patch (FIG. 3), sheets for application to a limb or torso or, a particular body region such as the thigh or waist, a sleeve, and the like.

With the exception of the patch, the substrate component 40 may be made by cutting a substrate into appropriate pieces such that the pieces, when attached to one another, form an appliance having an interior volume into which a portion of a body may be inserted. Patch configurations are sheets having a desired two-dimensional shape. See, for example the following references incorporated herein to the extent they are consistent with the present invention: U.S. Ser. No. 11/300,601 filed on Dec. 14, 2005 by Greene et al.; U.S. Ser. No. 11/590,445 filed on Dec. 15, 2006 by Bauer et al.; U.S. Ser. No. 11/601,033 filed in Oct. 31, 2006 by Johnson et al.; and U.S. Ser. No. 11/636,819 also filed on Oct. 31, 2006 by Johnson et al.

In general, a substrate member 40 that does not have an interior volume, such as a sheet or patch includes a substrate that is releasably attached to a backing. The substrate desirably contains a skin care composition configured to contact the skin of a user. Generally, the substrate can be a material onto which a formulation is applied, or in which it is integrated. See, U.S. Ser. No. 11/640,432 filed on Dec. 15, 2006 by Bauer et al., incorporated herein to the extent that it is consistent with the present invention.

In the embodiment illustrated in FIGS. 3 and 4, the skin care delivery device 110 is generally in the form of a mask. For instance, as shown, the substrate 112 includes a pair of spaced apart eye openings 116 and 118. The substrate 112 further includes an opening 120 designed to encircle the mouth of a user. If desired, the substrate 112 can further include a nose portion 22. The nose portion 122 can have any suitable configuration or shape so as to allow the mask to elevate over the nose of a user and/or allow a user to breathe through his or her nose.

The substrate 112 as shown in FIGS. 3 and 4 can be made from any suitable material capable of retaining a skin care composition. For example, the substrate can include a nonwoven fabric, woven fabric, knit fabric, wet-strength paper, or combinations thereof. Some particular examples of nonwoven fabrics that may used include, but are not limited to, spunbonded webs (apertured or non-apertured), meltblown webs, bonded carded webs, air-formed webs, coform webs, hydraulically entangled webs, and the like. In addition, nonwoven fabrics can contain synthetic fibers (e.g., polyethylenes, polypropylenes, polyvinyl chlorides, polyvinylidene chlorides, polystyrenes, polyesters, polyamides, polyimides, etc.); cellulosic fibers (softwood pulp, hardwood pulp, thermomechanical pulp, etc.); or combinations thereof.

If desired, the nonwoven fabric may also be bonded using techniques well known in the art to improve the durability, strength, hand, aesthetics, texture, and/or other properties of the fabric. For instance, the nonwoven fabric can be thermally (e.g., pattern bonded), ultrasonically, adhesively and/or mechanically (e.g., through-air dried) bonded. For instance, various pattern bonding techniques are described in U.S. Pat. No. 3,855,046 to Hansen; U.S. Pat. No. 5,620,779 to Levy, et al.; U.S. Pat. No. 5,962,112 to Haynes, et al.; U.S. Pat. No. 6,093,665 to Sayovitz, et al.; U.S. Design Pat. No. 428,267 to Romano, et al.; and U.S. Design Pat. No. 390,708 to Brown, which are incorporated herein in their entirety by reference thereto for all purposes.

In one embodiment of the present disclosure, the substrate 112 is formed from a nonwoven web containing a mixture of pulp fibers and synthetic fibers. Such substrates can be, for instance, hydroentangled webs or coform webs. Hydroentangling processes and hydroentangled composite webs containing various combinations of different fibers are known in the art. A typical hydroentangling process utilizes high pressure jet streams of water to entangle fibers and/or filaments to form a highly entangled consolidated fibrous structure, e.g., a nonwoven fabric.

The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein in their entirety by reference thereto for all purposes. Textured surfaces may be desirable for the manufacture of appliances that have an interior volume, such as a glove, sock, or other garment.

In another embodiment, the substrate 112 is formed from a spunbonded web containing monocomponent and/or multicomponent fibers. Multicomponent fibers are fibers that have been formed from at least two polymer components. Such fibers are usually extruded from separate extruders but spun together to form one fiber. The polymers of the respective components are usually different from each other although multicomponent fibers may include separate components of similar or identical polymeric materials. The individual components are typically arranged in substantially constantly positioned distinct zones across the cross-section of the fiber and extend substantially along the entire length of the fiber. The configuration of such fibers may be, for example, a side-by-side arrangement, a pie arrangement, or any other arrangement.

Of course, any of the substrates described above may be used on other part of the body, and are not intended to be limited to use on the face.

In addition to the substrate 112 described above, a substrate component 40 may be configured from a material that is imparted with texture on one or more surfaces. For instance, in some embodiments, the substrate can be formed from a dual-textured spunbond or meltblown material, such as described in U.S. Pat. No. 4,659,609 to Lamers, et al. and U.S. Pat. No. 4,833,003 to Win, et al., which are incorporated herein in their entirety by reference thereto for all purposes.

One example of a textured substrate, substrate 110, is depicted in FIG. 11, which representatively illustrates a water-impermeable layer 114 attached to an outer fibrous layer 112 and an inner fibrous layer 116. The material for the outer fibrous layer 112 may be any material that provides for a cloth-like appearance (as opposed to, for example, a smooth or rubbery appearance as in neoprene rubber glove). The material for the inner fibrous layer 116 may be any material that is fibrous in nature, such as a nonwoven material. The inner fibrous layer should possess an uneven, undulating surface to help contain the formulation or composition applied to the surface of the inner fibrous layer 116. As noted above, the rugosity of this inner material can be achieved or enhanced by attaching the inner fibrous layer 116 to the water-impermeable layer 114 at discrete points or locations (e.g., by thermally point bonding the materials together, as is discussed in more detail below) while the water-impermeable layer 114 is in a stretched condition. When the water-impermeable layer 114 (and, therefore, the resulting laminate) is allowed to relax, the inner fibrous layer 116 is gathered to produce undulations in the inner fibrous layer. Of course, both the inner fibrous layer 116 and the outer fibrous layer 112 are gathered in this way if they are attached to the water-impermeable layer 114 at discrete points or locations while the water-impermeable layer 114 is in a stretched condition (and then allowed to relax).

The inner and outer fibrous layers may be the same or may be different. Generally the water-impermeable layer 114 is elastomeric, with the resulting substrate 110 able to stretch and conform to a hand, foot, extremity, or other body region to which the appliance is applied.

The preceding paragraphs describe some examples of a substrate that may be used in an appliance of the present invention. However, any 3-layer substrate may be used, so long as the outer fibrous layer presents a cloth-like appearance and feel; the inner fibrous layer is sufficiently rugose to help contain the formulation or composition applied to the interior of the appliance (to minimize leakage of the formulation or composition from the appliance); and the water-impermeable layer—such as a film—is capable of minimizing evaporation or transfer of water through the appliance. For additional examples, see, e.g., U.S. Pat. No. 6,037,281, entitled “Cloth-Like, Liquid-Impervious, Breathable Composite Barrier Fabric,” to Mathis, et al.; U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; all of which are incorporated herein by reference in a manner consistent herewith.

Possible Formulations Used in Conjunction with the Substrate or Finishing Lotions

Formulations or compositions that can be used with any substrate or in any finishing lotion of the present invention may include emulsifiers, surfactants, viscosity modifiers, natural moisturizing factors, antimicrobial actives, pH modifiers, enzyme inhibitors/inactivators, suspending agents, pigments, dyes, colorants, buffers, perfumes, antibacterial actives, antifungal actives, pharmaceutical actives, film formers, deodorants, opacifiers, astringents, solvents, organic acids, preservatives, drugs, vitamins, aloe vera, and the like.

In one embodiment, a beneficial additive may be an emollient, which is herein defined as an agent that helps restore dry skin to a more normal moisture balance. Emollients act on the skin by supplying fats and oils that blend in with skin, making it pliable, repairing some of the cracks and fissures in the stratum corneum, and forming a protective film that traps water in the skin. Emollients that may be suitable for use with the present invention include beeswax, butyl stearate, cermides, cetyl palmitate, eucerit, isohexadecane, isopropyl palmitate, isopropyl myristate, mink oil, mineral oil, nut oil, oleyl alcohol, petroleum jelly or petrolatum, glyceral stearate, avocado oil, jojoba oil, lanolin (or woolwax), lanolin derivatives such as lanolin alcohol, retinyl palmitate (a vitamin A derivative), cetearyl alcohol, squalane, squalene, stearic acid, stearyl alcohol, myristal myristate, certain hydrogel emollients, various lipids, decyl oleate and castor oil.

A preferred beneficial additive is a humectant, which is herein defined to be an agent that supplies the skin with water by attracting moisture from the air and holding it on the skin. Humectants that may be suitable for use with the present invention include alanine, glycerin, PEG, propylene glycol, butylenes glycol, glycerin (glycol), hyaluronic acid, Natural Moisturizing Factor (a mixture of amino acids and salts that are among the skin's natural humectants), saccharide isomerate, sodium lactate, sorbitol, urea, and sodium PCA.

Other clinically beneficial agents that may be suitable for use with the present invention include antioxidants, a unique group of substances that protect a body or other objects from oxidizing. Antioxidants prevent or slow the oxidation process, thereby protecting the skin from premature aging. Exemplary antioxidants for use in the present invention include ascorbic acid ester, vitamin C (ascorbic acid), vitamin E (lecithin), Alpha-Glycosyl Rutin (AGR, or Alpha Flavon, a plant-derived antioxidant), and coenzyme Q10 (also known as ubiquinone).

Other clinically beneficial agents which may be delivered to the skin during use include chelating agents, such as EDTA; absorptive/neutralizing agents, such as kaolin, hectorite, smectite, or bentonite; other vitamins and vitamin sources and derivatives, such as panthenol, retinyl palmitate, tocopherol, and tocopherol acetate; anti-irritants such as chitin and chitosan.

Additional examples of beneficial agents include skin conditioners, which are herein defined as agents that may help the skin retain moisture, improve softness, or improve texture. Skin conditioners include, for example, amino acids, including alanine, serine, and glycine; allantoin, keratin, and methyl glucose dioleate; alpha-hydroxy acids, including lactic acid and glycolic acid, which act by loosening dead skin cells from the skin's surface; moisturizers (agents that add or hold water in dry skin), including echinacea (an extract of the coneflower plant), shea butter, and certain silicones, including cyclomethicon, dimethicone, and simethicone.

Other examples of beneficial botanical agents, extracts, or other materials that may be suitable for use with the present invention include almonds, chamomile extracts such as bisabolol (believed to relieve irritation, swelling and itching in the skin), elder flowers, honey, safflower oil, and elastin (safflower oil and elastin are believed to aid in retaining skin elasticity).

In addition to one or more clinically beneficial additives, other additives may be included in the formulation or composition. For example, a silicone polymer may be included to improve the slip characteristics of the elastomeric article. Possible silicone polymers include reactive silicones, non-reactive silicones, or a mixture of reactive and non-reactive silicones. Suitable silicones may include, for example, aminosilicones, polyether-modified amino silicones, amino-substituted siloxanes having terminal hydroxy groups, epoxy silicones, quaternary silicones, dimethicone, silicone polyethers, polyether epoxy silicones, silanol fluids, polysiloxy linoleyl pyrrolidone phospholipids, and combinations of possible silicones.

Other additives may be included, for example, glucose derived polymers, or mixtures containing glucose derived polymers (e.g., lauryl glucoside available from Cospha under the trade designation Planteran PS 400), silica, silica dispersions, wetting agents, and preservatives (i.e., parabens, such as methylparaben and propylparaben). In one embodiment, the personal-care composition may include emulsion stabilizers. Exemplary emulsion stabilizers include aluminum stearate, magnesium sulfate, hydrated silica, and ozokerite.

In another embodiment a beneficial agent may be held in the formulation or composition in liposomes. A liposome is a vehicle for delivering agents to the skin. More specifically, a liposome is a microscopic sphere formed from a fatty compound, a lipid, surrounding a water-based agent, such as a moisturizer or an emollient. When the liposome is rubbed into the skin, it releases the agent throughout the stratum corneum.

In another embodiment, the beneficial agent may be present in the carrier in the form of a microencapsulant. A microencapsulant is a sphere of an emollient surrounded by a gelatin membrane that prevents the emollient from reacting with other ingredients in the coating composition and helps distribute the emollient more evenly when pressure is applied and the membrane is broken. The process of forming these beads is called microencapsulation and is generally known in the art.

The formulation or composition of the present invention may be applied to the appliance as an aqueous solution, a dispersion, or an emulsion. In addition, the formulation or composition may be applied to the surface of the appliance as a micro-emulsion. A micro-emulsion is a particularly fine-particle emulsion that can be applied in a spray form.

The components of a formulation or composition may be applied in combination or separately to the surface of the appliance such as at the surface that is intended to be disposed directly against the skin. For example, a 100% humectant composition may be applied, followed by another 100% beneficial additive composition, such that the two (or more) separate applications together form the coating of the appliance. In such a manner, layers of additives may be built up on the surface of the appliance.

### Lotion Component 50:

The purpose of lotion component 50 is to place a layer of substance on the skin to retard water evaporation from the skin. This is why a lotion may also be referred to as a “moisturizer.” Naturally occurring skin lipids and sterols as well as artificial or natural oils, humectants, emollients, lubricants, etc. may be part of the composition of skin lotions or moisturizers. One representative example of a lotion component 50 includes one or more of the following ingredients: water, glycerin, mineral oil, dimethicone, preservatives, fragrance, and botanical extracts.

### Skin Lightening Component 60:

One possible purpose of skin lightening component 60 is to reduce the appearance of dark circles under the eye or on the skin. In one embodiment of the present invention, a skin lightening component 60 in lotion form suitable for use under the eye may contain the following ingredients: cetyl alcohol, laureth-23, glycolic acid, propylene glycol, isopropyl palmitate, mineral oil, glyceryl stearate, ozokerite, sodium hydroxide, water, steareth 20, tocopherol (vitamin E), dimethicone, xanthan gum, sodium PVM/MA decadiene crosspolymer, sorbic acid, sodium metabisulfite, imidazolidinyl urea, tetrasodium EDTA, fragrance, BHT. Other possible ingredients include: acetyl tetrapeptide-5, bis-hydroxyethyl tromethamine, buddleja davidii extract, butylene glycol, butylparaben, caffeine, camellia sinensis leaf extract, caprylic/capric triglyceride, ceratonia siliqua gum, chlorphenesin, citric acid, ethyl alcohol, ethylene/acrylic acid copolymer, ethylparaben, glycerin, glycosaminoglycans, witch hazel, hydroxyethylcellulose, thymus vulgaris (thyme) flower/leaf extract, panthenol, isobutylparaben, propylparaben, lecithin, methylparaben, phenoxyethanol, polysorbate 80, potassium sorbate, retinyl palmitate, sodium citrate, sodium hyaluronate, trisodium EDTA, and vitis vinifera (grape) fruit extract. The lotion may provided to the consumer in a packet, jar, bottle, or applicator. In one embodiment of the present invention, an applicator 62 has a swab configuration.

Other skin lightening components 60 may be used to reduce dark spots on the skin. Skin color comes from a natural pigment called melanin. Melanin pigment is synthesized in specialized cytoplasmic organelles called melanosomes. Within the melanosome, the enzymatic conversion of the amino acid tyrosine to melanin is catalyzed by the enzyme tyrosinase. Skin lightening compounds may be used to prevent such activity within the melansome. Skin lightening component 60 used for this purpose may include the following ingredients:
Alpha Arbutin: Alpha-Arbutin is a biosynthetic active ingredient that is pure, water-soluble and is manufactured in a powder form. It is the epimer of arbutin, and has shown to inhibit the activity of tyrosinase.
Beta-Arbutin (Bearberry Extract): Beta-Arbutin is often referred to as just Arbutin. Arbutin provides a skin lightening effect on the skin by inhibiting tyrosinase activity.
Kojic Acid Kojic acid is derived from a fungus, and studies have shown that it is effective as a lightening agent, inhibiting production of melanin (brown pigment).
Licorice Extract: The ingredient in licorice extract that is responsible for skin whitening is known as glabridin. Glabridin inhibits pigmentation by preventing tyrosinase activation. Licorice's anti-inflammatory properties (due to inhibition of superoxide anion production and cyclooxygenase activity) also make it a very popular ingredient in the skin care industry.
Niacinamide: Niacinamide is commonly known as Vitamin B3 and is an effective skin lightening compound that works by inhibiting melanosome transfer from melanocytes to keratinocytes.
Mulberry Extract Paper Mulberry extract, is obtained from the root of Broussonetia kazinoki, Siebold. or B. papyrifera, Vent. Tabl. Regn. Veget. or hybrids of both, family Moraceae. Extracts of this root are potent inhibitors of Tyrosinase enzyme. The active constituents present in the extract are Prenylated, polyhydroxylated mono- and bis-phenylderivatives.
Glycolic Acid: Glycolic Acid is an AHA (alpha hydroxy acid) which promotes exfoliation and a natural brightening of the skin tone. By encouraging cell turnover, glycolic acid not only evens out skin discolorations, but also helps to minimize fine lines and wrinkles. Glycolic Acid can assist other ingredients in skin lighteners by allowing them to penetrate farther into the skin.
Lactic Acid: Also an AHA, Lactic acid mimics the properties of Glycolic acid but is typically better suited for individuals with sensitive skin. AHA's such as Lactic Acid can assist other ingredients in skin lighteners by allowing them to penetrate farther into the skin.
Lemon Juice Extract: Lemon juice is one of nature's most potent skin bleaching ingredients. Unfortunately it can be irritating to the skin of some persons, and is used in small concentrations.
Vitamin C: Vitamin C is a natural antioxidant that occurs in many different forms (some stable and others unstable) each with distinct properties. Several of these forms have been shown to reduce melanin formation and provide a skin whitening effect when applied topically. These include 1-ascorbic acid, magnesium ascorbyl phosphate and sodium ascorbyl phosphate. These forms when used individually or together can assist in slowing down hyperactive melanocytes, resulting in lighter skin.

### Toner Component 70

A toner 70 may serve to reduce the appearance of pores. One possible toner formulation may include one or more of the following ingredients: water, butylene glycol, hamamelis witch hazel, glycerin, vitis vinifera fruit extract (grape), hydrolyzed vegetable protein, aloe barbadensis, hydrolized rice protein, salicylic acid, polysorbate 20, benzophenone 4, magnesium ascorbyl phosphate, diazolidinyl urea, methylparaben, propylparaben.

### Thermal Component 80

Some compounds, when applied to the skin, cause a cooling (endothermic) or heating (exothermic) effect. For example, an herbal such as menthol or peppermint oil applied to the skin causes the sensation of coolness, as does alcohol or butane. One possible cooling formulation may include one or more of the following ingredients: water, glycerin, cetearyl alcohol, petrolatum, cetyl alcohol, ceteareth 20, sodium lactate, lactic acid, peppermint oil, benzyl alcohol, diazolidinyl urea, menthol.

An herbal such as cinnamon or pepper (capsaicin) may cause a warm sensation, as does an exothermic reaction or an externally heated solution. One possible self-warming lotion may contain one or more of the following ingredients: propylene glycol, polyethylene glycol, lactic acid, hydroxypropylcellulose.

### Nail Conditioning Component 90

A nail conditioning component 90 contains ingredients that may prevent the nail and cuticle from drying out. One possible formulation contains one or more of the following ingredients: water, urea, sodium hypochlorite, glyceryl stearate SE, cetearyl alcohol, propylene glycol, mineral oil, simmondsia chinensis (Jojoba) seed oil, petrolatum, xanthan gum, DMDM hydantoin, methylparaben, propylparaben.

### Packaging Indicia

The package (not shown) may include indicia disposed at the outer surface thereof. The indicia may be printed, adhered, or otherwise disposed on surface for the purpose of providing an aesthetically pleasing design and/or to provide information to the user of components packaged in package. The indicia may include or solely contain messages, statements, or copy to be transmitted to a purchaser, consumer, or user of the components sold within package 10. In some embodiments, such messages, statements, or copy may be fashioned to help facilitate or establish an association in the mind of a user of the component one or more mental states, psychological states, or states of well being. The communication, statements, or copy may include various alphanumeric strings, including, for example: “rejuvenating,” “pampering,” “ritual,” “any time,” “clean,” “fresh,” “health,” “hygiene,” “well,” “well being,” “scent,” “convenient,” or “disposable,” derivatives or combinations thereof, or other such words or statements indicating that the components inside are capable of delivering to the user a spa-like experience. The indicia may further include aesthetic features such as a photograph and/or an artistic rendition of a subject relating to the component to be packaged in package, or a theme associated with the component. For example, a floral design may be used to imply freshness, or a person in a yoga pose may imply peace of mind. However, the present invention is not intended to be limited by these two random examples. It is further contemplated that such indicia may be printed on a device as well as a package, for example, the mask 40.

### Bonus Products

In addition to the system components that may be used in a step-by-step fashion, an additional bonus product may be added to the system 10 of the present invention. Such bonus products may not necessarily be directly related to the system 10. Instead, the bonus product may serve to introduce the user of system 10 to use another product, have an additional experience beyond the system 10, or entice a consumer to purchase another system 10 in the future. For example, items that may be bundled with one or more sets of a system 10 include: candles; towels (nonwoven or woven fabric); pillows; slippers; eye masks; music CDs; wearable fragrance or perfume; aromatic room freshener; coupons; vitamin supplements for ingestion; beverage items such as a tea bag, instant coffee, or a vitamin supplement preparation; dishwashing liquid; bodywash; toothpaste; mouthwash; or disposable razors. Of course, many other non-system related items may be included as a bonus product. In one embodiment, the bonus item may have a common theme with the system, such as a coordinating aesthetic feature (eg. a common design on system components and bonus products or their packaging).

Although only a few exemplary embodiments of this invention have been described in detail above, those skilled in the art will readily appreciate that many modifications are possible in the exemplary embodiments without materially departing from the novel teachings and advantages of this invention.

When introducing elements of the invention or the preferred aspect(s) thereof, the articles “a”, “an”, “the” and “said” are intended to mean that there are one or more of the elements. The terms “comprising”, “including” and “having” are intended to be inclusive and mean that there may be additional elements other than the listed elements.

1. A cosmetic system for the skin comprising:
a unit dose of a skin preparation compound in a first container;
a unit dose of a substrate comprising a sheet material with a skin care composition disposed thereon in a second container; and
a unit dose of a finishing lotion in a third container;
wherein the first, second, and third container are combined into a common package.

2. The cosmetic system of claim 1 wherein the skin preparation compound comprises an effervescent tablet that is dissolvable in water.

3. The cosmetic system of claim 1 wherein the skin preparation compound comprises an exfoliation component.

4. The cosmetic system of claim 1 wherein the exfoliation component comprises a buff.

5. The cosmetic system of claim 1 wherein the substrate is selected from a group consisting of a face mask, glove, and sock.

6. The cosmetic system of claim 1 wherein the exfoliation component comprises an exfoliation lotion.

7. The cosmetic system of claim 1 wherein the thermal compound comprises a substance selected from the group consisting of peppermint oil, alcohol, butane, and menthol.

8. The cosmetic system of claim 1 wherein the thermal compound comprises a substance selected from the group consisting of capsaicin, propylene glycol, polyethylene glycol, lactic acid, and hydroxypropylcellulose.

9. The cosmetic system of claim 1 further comprising instructions regarding the usage of the skin preparation compound, the substrate, and the finishing lotion.

10. A method of packaging a cosmetic system for the skin of a targeted body part, comprising the steps of:
a. disposing a unit dose of an exfoliation device in a first container to create an exfoliation component;
b. disposing a unit dose of a finishing lotion in a second container to create a lotion component;
c. disposing a unit dose of a substrate having a volume adapted to receive a body part into a third container to create a substrate component; and
wherein, the lotion component, the exfoliation component, and the substrate component together form a first component set which is disposed into a common container.

11. The method of claim 14, wherein steps a, b, and c are repeated to form a second component set, which is disposed into the common container.

12. The method of claim 15 wherein the first component set and the second component set is separated from one another within the common container.

13. The cosmetic system of claim 10 further including a bonus product.

14. The cosmetic system of claim 13 wherein the bonus product is a coupon.

15. The cosmetic system of claim 13 wherein the bonus product is a beverage item.

16. A cosmetic system for the skin comprising:
a skin exfoliation component;
a substrate comprising a sheet material and a hydrogel disposed thereon; and
a finishing lotion;
wherein the skin exfoliation component, the substrate, the lotion, and instructions for use of the same are combined into a common package.

17. The cosmetic system of claim 16 wherein the substrate comprises a sock.

18. The cosmetic system of claim 16 wherein the substrate comprises a glove.

19. The cosmetic system of claim 16 wherein the substrate comprises a face mask.

20. The cosmetic system of claim 16 wherein the skin exfoliation component comprises a buff.

21. The cosmetic system of claim 16 wherein the skin exfoliation component comprises a file.

22. The cosmetic system of claim 16 wherein the skin exfoliation component comprises a mechanical exfolient.

23. The cosmetic system of claim 16 wherein the skin preparation compound is an effervescent tablet.

24. The cosmetic system of claim 16 wherein the finishing lotion comprises a skin lightening component.

25. The cosmetic system of claim 16 wherein the finishing lotion comprises a skin moisturizer.

26. The cosmetic system of claim 16 wherein the finishing lotion comprises a thermal compound.

27. The cosmetic system of claim 16 wherein the finishing lotion comprises a toner.

28. The cosmetic system of claim 16 further comprising a nail conditioner.

29. A cosmetic system for the skin comprising:
a unit dose of skin preparation compound;
a substrate comprising a sheet material and a hydrogel disposed thereon; and
a unit dose of lotion;
wherein the skin preparation compound, the substrate, the lotion, and instructions for use of the same are combined into a common package.
